(19) United States
(12) Patent Application Publication
(10) Pub. No.: US 2025/0325820 A1
(43) Pub. Date: Oct. 23, 2025

(54) OFFSET PROCESSOR NOTIFICATION

(71) Applicant: Cochlear Limited, Macquarie University, NSW (AU)

(72) Inventors: Peter John Russell, Regentville (AU); Sabina Dabkowski-Chandler, Macquarie University, NSW (AU); Padraig Hurley, Macquarie University, NSW (AU); Jacqueline Goh, Macquarie University, NSW (AU); Shahana Moon, Macquarie University, NSW (AU); Shaun Ashwin Kumar, Rockdale, NSW (AU)

(21) Appl. No.: 18/870,225

(22) PCT Filed: Jul. 6, 2023
(86) PCT No.: PCT/IB2023/057014

§ 371 Date: Nov. 27, 2024

Related U.S. Application Data
(60) Provisional application No. 63/370,048, filed on Aug. 1, 2022.

(51) Int. Cl. A61N 1/372; A61N 1/36

(52) U.S. Cl. CPC A61N 1/37229; A61N 1/36038

(57) ABSTRACT

An apparatus includes a component configured to be placed over a portion of skin of a recipient, the portion of skin overlaying an implanted device. The apparatus further includes first circuitry within the component and configured to wirelessly communicate with second circuitry within the implanted device. The first circuitry has a set of optimal operational positions relative to the second circuitry. The apparatus further includes third circuitry configured to detect at least one parameter indicative of a displacement of the first circuitry from the set of optimal operational positions and to generate at least one signal indicative of a magnitude and/or direction for moving the first circuitry towards the set of optimal operational positions.

### BACKGROUND

### Field

The present application relates generally to implantable medical systems, and more specifically to systems having an external portion and an implanted portion configured to transcutaneously wirelessly communicate with one another.

### Description of the Related Art

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, cochlear implants, etc.), implantable pacemakers, defibrillators, functional electrical stimulation devices, and other implantable medical devices, have been successful in performing lifesaving and/or lifestyle enhancement functions and/or recipient monitoring for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, the implantable medical device.

### SUMMARY

In one aspect disclosed herein, an apparatus comprises a component configured to be placed over a portion of skin of a recipient, the portion of skin overlaying an implanted device. The apparatus further comprises first circuitry within the component. The first circuitry is configured to wirelessly communicate with second circuitry within the implanted device. The first circuitry has a set of optimal operational positions relative to the second circuitry. The apparatus further comprises third circuitry configured to detect at least one parameter indicative of a displacement of the first circuitry from the set of optimal operational positions and to generate at least one signal indicative of a magnitude and/or direction for moving the first circuitry towards the set of optimal operational positions.

In another aspect disclosed herein, a method comprises prompting a user to sequentially locate an external device outside a recipient's body at a series of positions relative to an internal device within the recipient's body. The external device comprises at least one external communication coil and the internal device comprising at least one internal communication coil. The method further comprises receiving data indicative of measured coupling values between the at least one external communication coil and the at least one internal communication coil. The measured coupling values is measured with the at least one external communication coil located at different positions of the series of positions. The method further comprises determining an optimal position of the external device. The optimal position corresponds to a maximum coupling value obtained using the measured coupling values.

In still another aspect disclosed herein, a method comprises receiving at least one first measured value of inductive coupling between an external device outside a recipient's body and an internal device within the recipient's body. The method further comprises accessing data corresponding to second measured values of inductive coupling between other external devices outside other recipients' bodies and other internal devices within the other recipients' bodies. The method further comprises comparing the first measured value to the second measured values. The method further comprises generating, in response to said comparing, an evaluation of the inductive coupling between the external device and the internal device

In still another aspect disclosed herein, a system comprises a component having an outer surface portion configured to be placed over a portion of skin of a recipient, the portion of skin overlaying an implanted device. The system further comprises a first magnet within the housing, the first magnet comprising a first substantially flat magnetic surface configured to be substantially parallel to the portion of skin and having a first north magnetic pole region and a first south magnetic pole region. The implanted device comprises a second magnet having a second substantially flat magnetic surface substantially parallel to the portion of skin and having a second north magnetic pole region and a second south magnetic pole region. The system further comprises circuitry within the housing. The circuitry is configured to detect a configuration of the first magnet and the second magnet upon the component being placed at least partially over the implanted device and to generate at least one signal indicative of the configuration.

In still another aspect disclosed herein, a method comprises placing an external component of a medical device on skin of a recipient such that only one of two magnetic poles of the external component overlays a corresponding one of two magnetic poles of an internal component of the medical device implanted in the recipient. The method further comprises receiving one or more prompts originating from the medical device to adjust a placement of the external component on the skin of the recipient until both of the two magnetic poles of the external component overlay both of the corresponding two magnetic poles of the internal component.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 schematically illustrates a cross-sectional view of an example apparatus in accordance with certain implementations described herein;

FIGS. 3A and 3B schematically illustrate two perspective views of an example at least one first magnet and an at least one example second magnet in accordance with certain implementations described herein;

FIG. 3C schematically illustrates side views of four example magnets that are compatible for use as the at least one first magnet and/or the at least one second magnet in accordance with certain implementations described herein;

FIG. 4 schematically illustrates an example apparatus in two possible configurations in accordance with certain implementations described herein;

FIG. 5 is a flow diagram of an example method in accordance with certain implementations described herein;

FIGS. 6A and 6B schematically illustrate example images shown on a user interface of a communication device visible to the user during the method in accordance with certain implementations described herein;

FIG. 6C schematically illustrates a baseline mapping using the data received during the method in accordance with certain implementations described herein;

FIGS. 7A and 7B schematically illustrate example images shown on a user interface of a communication device visible to the user while positioning the external device in accordance with certain implementations described herein;

FIG. 8 is a flow diagram of an example method in accordance with certain implementations described herein; and

FIG. 9 is a flow diagram of another example method in accordance with certain implementations described herein.

### DETAILED DESCRIPTION

Certain implementations described herein a system and method by which a medical device (e.g., auditory prosthesis) having an implanted portion configured to transcutaneously wirelessly communicate (e.g., via an RF link) with an external portion via magnetic induction coils. The medical device is able to detect and to clearly notify a user (e.g., recipient; practitioner) when the external portion is placed improperly (e.g., laterally offset; angularly offset; eccentrically; not ideally aligned) relative to an optimal placement (e.g., concentric and parallel), adversely affecting the transcutaneous wireless communication. Information indicative of the relative displacement between the external and internal portions can be obtained from measurements indicative of the link integrity and/or efficiency and can be presented to the user as feedback information for positioning the external portion to achieve improved coupling. By facilitating avoidance of sub-optimal coil placements, the battery life can be enhanced, and the recipient can experience fewer signal cut-outs during operation of the medical device. In addition, the recipient can be made more aware of the device performance and can take steps to improve the device performance. Furthermore, comparisons of data regarding the recipient's coupling to such data for other recipients can be used to generate guidance (e.g., recommendations) for improving the recipient's link integrity and efficiency.

The teachings detailed herein are applicable, in at least some implementations, to any type of implantable medical device, for example, auditory prosthesis utilizing an implantable actuator assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of medical device that can utilize the teachings detailed herein and/or variations thereof.

Merely for ease of description, apparatus and methods disclosed herein are primarily described with reference to an illustrative medical device, namely an active transcutaneous or percutaneous auditory prosthesis system. Implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses. However, the teachings detailed herein and/or variations thereof may also be used with a variety of other medical or non-medical systems (e.g., other types of devices beyond auditory prostheses) that provide a wide range of therapeutic benefits to recipients, patients, or other users. For example, the concepts described herein can be applied to any of a variety of implantable medical devices that utilize the transfer of power and/or data between an implanted component and an external component via inductive coupling (e.g., pacemakers; implantable EEG monitoring devices; implantable seizure monitoring devices; visual prostheses).

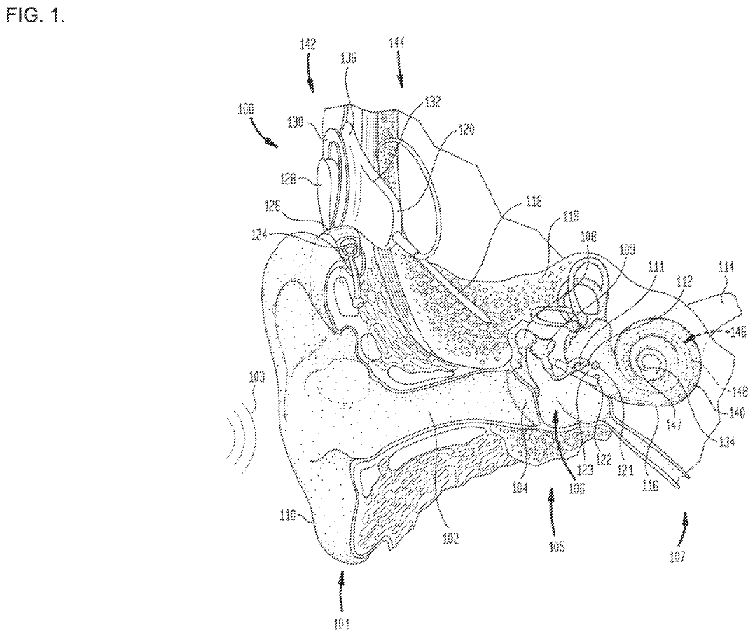

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain implementations described herein can replace the external microphone assembly 124 shown in FIG. 1 with a subcutaneously implantable assembly comprising an acoustic transducer (e.g., microphone), as described more fully herein.

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more input elements/devices for receiving input signals at a sound processing unit 126. The one or more input elements/devices can include one or more sound input elements (e.g., one or more external microphone assemblies 124) for detecting sound and/or one or more auxiliary input devices (not shown in FIG. 1) (e.g., audio ports, such as a Direct Audio Input (DAI); data ports, such as a Universal Serial Bus (USB) port; cable ports, etc.). In the example of FIG. 1, the sound processing unit 126 is a behind-the-ear (BTE) sound processing unit configured to be attached to, and worn adjacent to, the recipient's ear. However, in certain other implementations, the sound processing unit 126 has other arrangements, such as by an OTE processing unit (e.g., a component having a generally cylindrical shape and which is configured to be magnetically coupled to the recipient's head), etc., a mini or micro-BTE unit, an in-the-canal unit that is configured to be located in the recipient's ear canal, a body-worn sound processing unit, etc.

The sound processing unit 126 of certain implementations includes a power source (not shown in FIG. 1) (e.g., battery), a processing module (not shown in FIG. 1) (e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementations of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., external microphone assembly 124 that is positioned externally to the recipient's body, in the depicted embodiment of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing, sometimes collectively referred to as a stimulator/receiver unit. The internal receiver unit 132 comprises at least one internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 can be implanted at least in the basal region 116, and sometimes further. For example, the electrode assembly 118 can extend towards an apical end of the cochlea 140, referred to as the cochlea apex 134. In certain circumstances, the electrode assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate electrode assembly 118 comprises a longitudinally aligned and distally extending array 146 of electrodes 148 (e.g., contacts), sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

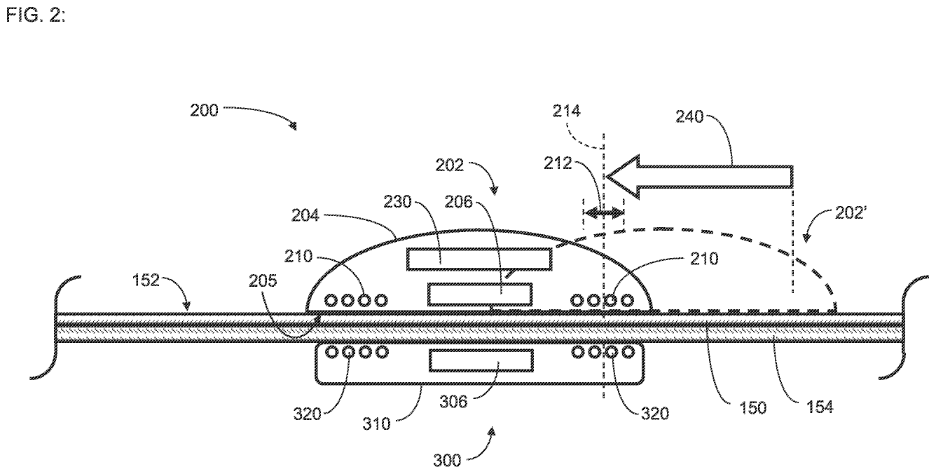

FIG. 2 schematically illustrates a cross-sectional view of an example apparatus 200 in accordance with certain implementations described herein. The apparatus 200 comprises a component 202 configured to be placed over a portion of skin 150 of a recipient, the portion of skin 150 overlaying an implanted device 300. The apparatus 200 further comprises first circuitry 210 within the component 202, the first circuitry 210 configured to wirelessly communicate with second circuitry 320 within the implanted device 300. The first circuitry 210 has a set of optimal operational positions 212 relative to the second circuitry 320. The apparatus 200 further comprises third circuitry 230 configured to detect at least one parameter indicative of a displacement of the first circuitry 210 from the set of optimal operational positions 212 and to generate at least one signal indicative of a magnitude and/or direction 240 for moving the first circuitry 210 towards the set of optimal operational positions 212.

In certain implementations, the component 202 is an external component 142 of an implantable medical device (e.g., an auditory prosthesis system; a cochlear implant auditory prosthesis 100 as schematically illustrated by FIG. 1), and the implanted device 300 is an internal component 144 of the implantable medical device. For example, the component 202 can be an external component 142 of an auditory prosthesis system selected from the group consisting of: a cochlear implant system, a Direct Acoustic Cochlear Implant (DACI) system, a middle ear implant system, a middle ear transducer (MET) system, an electro-acoustic implant system, another type of auditory prosthesis system, and/or combinations or variations thereof.

In certain implementations, the component 202 comprises a housing 204 (e.g., comprising a polymer material and/or other material compatible for being placed in contact with a surface 152 of the recipient's skin 150) containing the first circuitry 210 (e.g., an external transmitter unit 128 comprising at least one external inductive communication coil 130) and the third circuitry 230 as well as other elements of the component 202 (e.g., at least one external microphone assembly 124, a sound processing unit 126, and/or a power source). For example, the first circuitry 210, the third circuitry 230, and other elements of the component 202 can be contained within one or more cavities (e.g., hermetically sealed regions) of the housing 204. In certain implementations, the housing 204 comprises an outer surface portion 205 configured to be placed over and substantially parallel to the portion of skin 150.

In certain implementations, the implantable device 300 comprises an implantable housing 310 (e.g., comprising titanium, polymer, ceramic, or other biocompatible material compatible for being implanted beneath the recipient's skin 150 and other tissue 154 such as muscle and/or fat) containing the second circuitry 320 (e.g., at least one internal inductive communication coil 136) as well as other elements of the implantable device 300 (e.g., an implanted receiver unit 132, a stimulator unit 120, and/or an elongate electrode assembly 118). For example, the second circuitry 320 and other elements of the implantable device 300 can be contained within one or more cavities (e.g., hermetically sealed regions) of the implantable housing 310. In certain implementations, the implantable housing 310 of the implantable device 300 is configured to be affixed to a bone surface (e.g., skull surface) beneath the skin 150 and other tissue 154.

In certain implementations, the apparatus 200 further comprises at least one first magnet 206 mounted within the component 202 (e.g., within a cavity of the housing 204) and the implantable device 300 comprises at least one second magnet 306 within the implanted device 300 (e.g., hermetically sealed within the implantable housing 310). The at least one first magnet 206 can be configured to generate an attractive magnetic force with the at least one second magnet 306 within the implanted device 300. The attractive force can be configured to hold the component 202 on the surface 152 of the portion of skin 150 with the first circuitry 210 at the set of optimal operational positions 212. Magnets compatible with certain implementations described herein include, but are not limited to, axially magnetized ferromagnets (e.g., axial magnets) and diametrically magnetized ferromagnets (e.g., diametric magnets). In certain other implementations, the component 202 and/or the implanted device 300 does not contain a magnet and the component 202 is held on the surface 152 of the portion of skin 150 by other forces (e.g., from external pressure applied to the component 202), and the apparatus 200 provides guidance regarding the optimal external coil placement.

In certain implementations, the first circuitry 210 comprises a first planar inductor coil (e.g., the at least one external inductive communication coil 130 of the external transmitter unit 128) having a first number of turns and a first planar area and the second circuitry 320 comprises a second planar inductor coil (e.g., the at least one internal inductive communication coil 136 of the internal receiver unit 132) having a second number of turns and a second area. The first planar inductor coil can be configured to be in inductive communication with the second planar inductor coil. In certain implementations in which the component 202 comprises at least one first magnet 206 and the implantable device 300 comprises at least one second magnet 306, the first planar inductor coil can encircle the at least one first magnet 206 and/or a projection of the at least one first magnet 206 onto a plane defined by the first planar inductor coil, and the second planar inductor coil can encircle the at least one second magnet 306 and/or a projection of the at least one second magnet 306 onto a plane defined by the second planar inductor coil.

In certain implementations, the first circuitry 210 and the second circuitry 320 form a transcutaneous inductive radio frequency (RF) communication link between the apparatus 200 and the implanted device 300 (e.g., the first circuitry 210 and the second circuitry 320 interact with one another via magnetic flux of one of the first and second circuitry 210, 320 passing through the other one of the first and second circuitry 210, 320), across which the implanted device 300 receives power and/or data signals from the apparatus 200. In certain implementations, when the apparatus 200 is placed in an operational position, the first circuitry 210 is substantially centered over the second circuitry 320. For example, a center axis of the first circuitry 210 can be substantially coincident with a center axis of the second circuitry 320.

In certain implementations, the inductive coupling (e.g., mutual inductance; coupling coefficient) between the first circuitry 210 and the second circuitry 320 is dependent upon the displacement between the first circuitry 210 and the second circuitry 320, with larger displacements corresponding to weaker inductive coupling. For example, larger displacements in a direction substantially perpendicular to the skin (e.g., larger skin flap thicknesses or SFT) can reduce the inductive coupling between the first circuitry 210 and the second circuitry 320, and larger displacements in directions substantially parallel to the skin 150 (e.g., lateral displacements) can also reduce the inductive coupling between the first circuitry 210 and the second circuitry 320. In addition, the inductive coupling can be degraded by angular displacement (e.g., tilt) between the first circuitry 210 and the second circuitry 320.

Certain implementations described herein have a set (e.g., range) of positions (referred to herein as a set of optimal operational positions 212) at which the inductive coupling between the first circuitry 210 and the second circuitry 320 is optimal for operation of the apparatus 200 (e.g., sufficiently efficient operational signal transmission between the component 202 and the implanted device 300). For example, as shown in FIG. 2, the first circuitry 210 can be positioned directly over the second circuitry 320 (denoted in FIG. 2 by the dashed line 214) providing a maximal overlap of the first area and the second area and a maximal inductive coupling that provides maximally efficient signal transmission between the component 202 and the implanted device 300. However, the first circuitry 210 of certain implementations can be displaced (e.g., laterally displaced in a direction substantially parallel to the portion of skin 150; axially displaced in a direction substantially perpendicular to the portion of skin 150; angularly displaced to have a tilt relative to the portion of skin 150) from this maximally coupled position. For small displacements, the inductive coupling is less than the maximal inductive coupling but is still adequate for sufficiently efficient signal transmission between the component 202 and the implanted device 300. For large displacements (e.g., denoted in FIG. 2 by the dashed component 202′), the inductive coupling is significantly less than the maximal inductive coupling and is inadequate for sufficiently efficient signal transmission. In certain implementations, a range of the inductive coupling (e.g., mutual inductance; coupling coefficient) values that are adequate for sufficiently efficient signal transmission is predetermined and the set of optimal operational positions 212 corresponds to this range of inductive coupling values. For example, a predetermined threshold inductive coupling value can separate the values for sufficiently efficient signal transmission from the values for insufficiently efficient signal transmission.

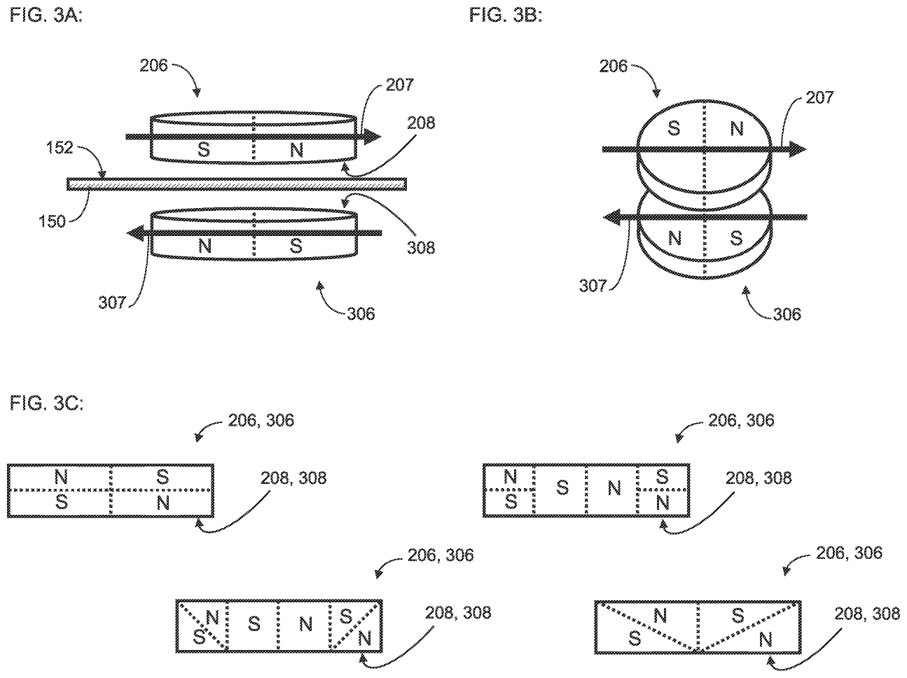

FIGS. 3A and 3B schematically illustrate two perspective views of an example at least one first magnet 206 and an example at least one second magnet 306 in accordance with certain implementations described herein. FIG. 3C schematically illustrates side views of four example magnets (e.g., “4-pole” magnet; “Halbach” magnet; “angled Halbach” magnet; “angled 4-pole” magnet) that are compatible for use as the at least one first magnet 206 and/or the at least one second magnet 306 in accordance with certain implementations described herein. The at least one first magnet 206 can comprise a diametric magnet affixed on or within the housing 204 and having a first magnetic moment 207 (e.g., magnetization) extending substantially parallel to the outer surface portion 205 (e.g., substantially parallel to the portion of skin 150 when the first circuitry 210 is at the set of optimal operational positions 212). The at least one second magnet 306 can comprise a diametric magnet within the implantable housing 310 and having a second magnetic moment 307 (e.g., magnetization) extending substantially parallel to the portion of skin 150. In the context of auditory prostheses, implanted devices 300 with rotatable diametric implanted magnets 306 can provide compatibility with magnetic resonance imaging (MRI) by having the implanted magnet 306 rotate about its center axis in response to the torque induced by the large MRI magnetic fields interacting with the implanted magnet 306, thereby avoiding pain and/or damage to the recipient and/or the auditory prosthesis.

Various configurations of the at least one first magnet 206 and the at least one second magnet 306 are also compatible with certain implementations described herein. For example, the at least one second magnet 306 can comprise a plurality of cylinders configured to rotate, with magnetic attraction between the cylinders greater than magnetic attraction to the at least one first magnet 206 (e.g., the at least one second magnet 306 functioning like a diametrically magnetized magnet, except in the presence of an MRI field, which can be very strong and can overcome the cylinder-to-cylinder magnetic attraction). For another example, the at least one first magnet 206 and/or the at least one second magnet 306 can each comprise a first portion having a first magnetization and a second portion having a second magnetization having a non-zero oblique angle or an orthogonal angle relative to the first magnetization. In certain implementations, the set of optimal operational positions 212 of the first circuitry 210 corresponds to a non-zero offset between the at least one first magnet 206 and the at least one second magnet 306.

As shown in FIGS. 3A and 3B, the at least one first magnet 206 of certain implementations comprises a unitary (e.g., monolithic) body with two half portions (e.g., two semicircular portions) comprising a single first north pole (labeled “N”) and a single first south pole (labeled “S”). The at least one first magnet 206 of certain other implementations comprises a plurality of first north poles and a plurality of first south poles (see, e.g., FIG. 3C). The at least one first magnet 206 of certain implementations comprises a substantially flat outer surface 208 (e.g., lower surface closest to the portion of skin 150) configured to be substantially parallel to the portion of skin 150 and having a single first north magnetic pole region and a single first south magnetic pole region (see, e.g., FIGS. 3A-3C).

Similarly, as shown in FIGS. 3A and 3B, the at least one second magnet 306 of certain implementations comprises a unitary (e.g., monolithic) body with two half portions (e.g., two semicircular portions) comprising a single second north pole (labeled “N”) and a single second south pole (labeled “S”). The at least one second magnet 306 of certain other implementations comprises a plurality of second north poles and a plurality of second south poles (see, e.g., FIG. 3C). The at least one second magnet 306 of certain implementations comprises a substantially flat outer surface 308 (e.g., upper surface closest to the portion of skin 150) configured to be substantially parallel to the portion of skin 150 and having a single second north magnetic pole region and a single second south magnetic pole region.

Each of the at least one first magnet 206 and the at least one second magnet 306 can comprise at least one ferromagnetic material selected from the group consisting of: iron, nickel, cobalt, neodymium, and steel. While FIGS. 3A-3C show the at least one first magnet 206 and the at least one second magnet 306 having a right circular cylindrical shape with a center axis substantially perpendicular to the respective magnetic moment, other shapes (e.g., circular; elliptical; square; rectangular; polygonal; geometric; irregular; symmetric; non-symmetric; with straight, curved, or irregular sides) are also compatible with certain implementations described herein. In certain implementations, as shown in FIGS. 3A-3C, one or both of the at least one first magnet 206 and the at least one second magnet 306 is a unitary (e.g., monolithic) magnet, while in certain other implementations, one or both of the at least one first magnet 206 and the at least one second magnet 306 each comprises a plurality of magnets.

In certain implementations, the attractive magnetic force between the at least one first magnet 206 and the at least one second magnet 306 facilitates the positioning of the component 202 relative to the implanted device 300. For example, when the component 202 is moved sufficiently close to the implanted device 300 to generate the attractive magnetic force, the attractive magnetic force acts to position the component 202 such that the at least one first magnet 206 is directly over the at least one second magnet 306, with the first magnetic moment 207 substantially parallel to and opposite to the second magnetic moment 307 (e.g., as schematically illustrated by FIGS. 3A and 3B). The fixed position of the first circuitry 210 relative to the at least one first magnet 206 within the component 202 and the fixed position of the second circuitry 320 relative to the at least one second magnet 306 within the implanted device 300 can be configured such that the attractive magnetic force provides sufficient retention to keep the component 202 on the surface 152 of the portion of skin 150 with the first circuitry 210 being at a position within the set of optimal operational positions 212 relative to the second circuitry 320 (e.g., the first circuitry 210 centered over and/or concentric with the second circuitry 320). While FIGS. 3A and 3B schematically illustrate each of the at least one first magnet 206 and the at least one second magnet 306 comprising a diametric magnet with the corresponding first and second magnetic moments 207, 307 extending substantially parallel to the portion of skin 150, in certain other implementations, each of the at least one first magnet 206 and the at least one second magnet 306 comprises an axial magnet with the corresponding first and second magnetic moments 207, 307 extending substantially perpendicularly to the portion of skin 150.

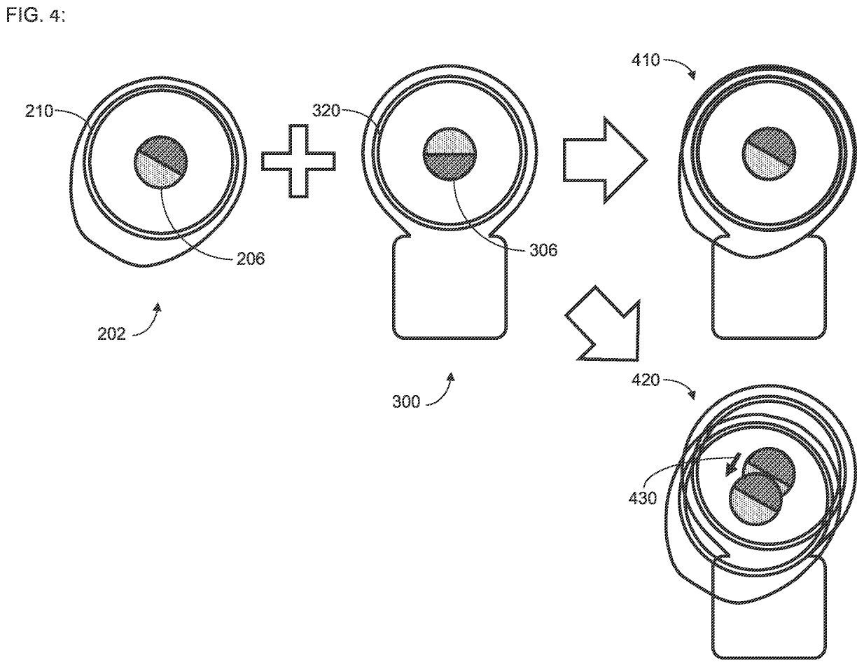

FIG. 4 schematically illustrates an example apparatus 200 with the at least one first magnet 206 and the at least one second magnet 306 in two possible configurations in accordance with certain implementations described herein. The at least one first magnet 206 of the component 202 comprises a first diametric magnet with north and south poles (e.g., see FIGS. 3A-3B; shown in FIG. 4 with different shadings) and the at least one second magnet 306 of the implanted device 300 comprises a second diametric magnet with north and south poles (e.g., see FIGS. 3A-3B; shown in FIG. 4 with different shadings). The at least one second magnet 306 of FIG. 4 is configured to freely rotate within a plane substantially parallel to the portion of skin 150 while remaining within the implantable housing 310 of the implanted device 300 (e.g., rotate about a rotation axis substantially perpendicular to the portion of skin 150).

In a first configuration 410 (shown in the upper right of FIG. 4), the component 202 is placed over the implanted device 300 with the at least one first magnet 206 directly over (e.g., concentric with; axially aligned with) the at least one second magnet 306 of the implanted device 300. While not visible in FIG. 4, the at least one second magnet 306 has rotated in a plane substantially parallel to the portion of skin 150 such that the second north pole is directly beneath the first south pole and the second south pole is directly beneath the first north pole (e.g., providing a maximal retention force). In this first configuration 410, the first circuitry 210 is directly over (e.g., concentric with; axially aligned with) the second circuitry 320, thereby providing a maximal inductive coupling between the first circuitry 210 and the second circuitry 320.

However, in other configurations, the component 202 can be placed over the implanted device 300 with a displacement 430 of the first circuitry 210 from the set of optimal operational positions 212 (e.g., the first configuration 410). For example, the at least one second magnet 306 can rotate while the component 202 is being placed over the implanted device 300, and, as shown in example second configuration 420 in the lower right of FIG. 4, the at least one first magnet 206 can be laterally offset from (e.g., non-concentric with; axially misaligned with) the at least one second magnet 306, with a corresponding lateral displacement (e.g., offset) of the first circuitry 210 from the second circuitry 320. In this example second configuration 420, only one of the two poles of the at least one second magnet 306 is directly beneath the opposite pole of the at least one first magnet 206 (e.g., providing some retention force but less than the maximal retention force), and the first circuitry 210 is laterally offset from (e.g., non-concentric with; axially misaligned with) the second circuitry 320, thereby providing less than the maximal inductive coupling between the first circuitry 210 and the second circuitry 320. While FIG. 4 schematically illustrates one example second configuration 420 having a displacement 430 comprising a lateral displacement (e.g., offset) in a direction substantially parallel to the portion of skin 150, the displacement 430 of other second configurations 420 compatible with certain implementations described herein can include an axial displacement (e.g., in a direction substantially perpendicular to the portion of skin 150) of the first circuitry 210 from the second circuitry 320 and/or an angular displacement (e.g., tilt) between the first circuitry 210 and the second circuitry 320.

In certain implementations, it can be difficult for a recipient or a user placing the component 202 in operative position relative to the implanted device 300 (which is beneath the skin 150) to distinguish whether the component 202 is properly positioned relative to the implanted device 300 (e.g., to distinguish between the first configuration 410 which provides the maximal or optimal inductive coupling and the second configuration 420 which provides a smaller inductive coupling). An apparatus 200 with merely a simple binary notification scheme (e.g., LED indication with on/off corresponding to good/bad positioning) may not be able to reliably distinguish the less-desirable second configuration 420 from the optimal first configuration 410. In addition, positioning procedures for avoiding the second configuration 420 can be complicated and difficult for recipients to follow. Operating the apparatus 200 in the second configuration 420 with the smaller retention force and smaller inductive coupling can result in various issues, including but not limited to: reduced battery life of the component 202; dissatisfaction with the retention of the component 202 on the surface 152 of the portion of skin 150; communication errors between the component 202 and the implanted device 300 resulting in loss of operation.

Furthermore, in the context of a surgical use case in which a newly inserted implanted device 300 is tested prior to closing the surgical site, even though the implanted device 300 may not be obscured by tissue, it can be difficult for a practitioner (e.g., surgeon; nurse) to know when the first circuitry 210 of the component 202 (e.g., testing device) is properly aligned with the second circuitry 320 of the implanted device 300, causing frustration and delays during surgery. Optimal positioning of the first circuitry 210 can be constrained by surgical drapings and/or increased SFT ranges due to tissue swelling can increase the effective coil-to-coil separation by up to and beyond 16 millimeters, and misalignments can increase the effective separation further. For example, a longitudinal offset between the first circuitry 210 and the second circuitry 320 of 5 millimeters can be equivalent to about 3 millimeters of additional SFT, an angular offset between the first circuitry 210 and the second circuitry 320 of 15 degrees can be equivalent to about 5 millimeters of additional SFT, and a combination of these longitudinal and angular offsets can be equivalent to about 7 millimeters of additional SFT. Magnets cannot be used in all such cases as a measure of optimal coil alignment. A wireless communication link able to operate to about 23 millimeters of total effective separation and without certain implementations described herein might provide optimal user experience during surgical use. However, certain implementations described herein are configured to provide optimal user experience with a wireless communication link able to operate to about 10 millimeters and/or to improve the ease and accuracy of positioning of the testing device (e.g., to reduce the likelihood of frustration and/or delays).

In certain implementations, the third circuitry 230 is configured to detect at least one parameter indicative of the displacement 430 and to provide guidance information to the user (e.g., recipient; practitioner) to facilitate proper operational positioning of the component 202 relative to the implanted device 300. In certain implementations, as schematically illustrated by FIG. 2, the third circuitry 230 is within the component 202, while in certain other implementations, the third circuitry 230 is within the implanted device 300 or within a device separate from both the component 202 and the implanted device 300 (e.g., smartphone, smart tablet, smart watch, or other remote device operated by the user and in communication with the component 202 and/or the implanted device 300). The third circuitry 230 of certain implementations comprises at least one microcontroller configured to receive detection signals indicative of the at least one parameter and to generate output signals in response. The at least one microcontroller can comprise at least one application-specific integrated circuit (ASIC) microcontroller, digital signal processing (DSP) microcontroller, generalized integrated circuits programmed by software with computer executable instructions, and/or microcontroller core. In certain implementations, the third circuitry 230 and the first circuitry 210 comprise different portions of the same circuitry (e.g., a single microcontroller), while in certain other implementations, the third circuitry 230 and the first circuitry 210 comprise portions of different microcontrollers. In certain implementations, the third circuitry 230 comprises and/or is in operative communication with storage circuitry configured to store information (e.g., data; commands) accessed by the third circuitry 230 during operation (e.g., while providing the functionality of certain implementations described herein). The storage circuitry can comprise at least one tangible (e.g., non-transitory) computer readable storage medium, examples of which include but are not limited to: read only memory (ROM); random access memory (RAM); magnetic disk storage media; optical storage media; flash memory. The storage circuitry can be encoded with software (e.g., a computer program downloaded as an application) comprising computer executable instructions for instructing the third circuitry 230 (e.g., executable data access logic, evaluation logic, and/or information outputting logic). In certain implementations, the third circuitry 230 executes the instructions of the software to provide functionality as described herein. The third circuitry 230 of certain implementations further comprises other digital circuitry (e.g., registers; filters; output controllers; memory controllers).

In certain implementations, the at least one parameter comprises a magnetic field strength of the attractive magnetic field between the at least one first magnet 206 and the at least one second magnet 306. For example, the third circuitry 230 can comprise a magnetic field detection circuit (e.g., Hall effect sensor) configured to generate detection signals indicative of the strength of the attractive magnetic field.

In certain implementations, the at least one parameter is indicative of efficiency and/or data integrity of communications between the first circuitry 210 and the second circuitry 320. In certain implementations, the at least one parameter comprises a mutual inductance of the first circuitry 210 (e.g., the at least one external inductive communication coil 130) and the second circuitry 320 (e.g., the at least one internal inductive communication coil 136), a power consumption rate of communications between the first circuitry 210 and the second circuitry 320, a voltage or decay rate received by the implanted device 300, or a radio-frequency (RF) communication efficiency between the first circuitry 210 and the second circuitry 320. For example, the third circuitry 230 can be configured to receive detection signals (e.g., from the first circuitry 210) indicative of the input voltage, current, and/or power provided to the first circuitry 210 and/or to receive detection signals (e.g., from the second circuitry 320 via backlink telemetry) indicative of the output voltage, current, and/or power received by the second circuitry 320. In certain implementations, the at least one parameter comprises a number or rate of communication errors between the first circuitry 210 and the second circuitry 320 (e.g., cyclic redundancy check or CRC of signals transmitted between the first circuitry 210 and the second circuitry 320 via backlink telemetry). For example, one of the first circuitry 210 and the second circuitry 320 can transmit test communication signals to the other of the first circuitry 210 and the second circuitry 320, and the third circuitry 230 can detect the number and/or rate of communication errors of these transmitted test communication signals (e.g., over a predetermined time period). Certain such implementations can be configured in firmware and/or software of the apparatus 200, without additional hardware.

In certain implementations, the apparatus 200 comprises a user interface configured to generate audio, visual, and/or haptic indicia, in response to the at least one signal, to communicate the magnitude and/or direction for moving the first circuitry 210 towards the set of optimal operational positions 212 to the user. These indicia can be configured to provide the user with information (e.g., feedback indicative of the relative alignment compared to a previous baseline and guidance for improving the alignment) to be used by the user in deciding whether the component 202 should be moved to improve the coupling of the first circuitry 210 and the second circuitry 320. This information can be in the form of a hotter/colder indication (e.g., hotter corresponding to closer to optimal positioning and colder corresponding to farther from optimal positioning) or other type of indication. For example, the at least one signal and/or the indicia can be indicative of the magnitude being non-zero (e.g., such that movement of the first circuitry 210 is warranted). For another example, the measured values of the at least one parameter can be converted to scores and ranges of these scores can correspond to poor, average, or optimal connections between the first circuitry 210 and the second circuitry 320, with the present score being displayed to the user. Upon the user interface indicating that an optimal operational position has been achieved, the user could then know to hold the component 202 and the first circuitry 210 in place.

For example, the user interface can comprise a light source (e.g., one or more light emitting diodes) configured to exhibit colors and/or to flash at rates indicative of the magnitude and/or direction. When the first circuitry 210 is farther from the set of optimal operational positions 212, the light source can emit a first color (e.g., red) and/or can flash at a slow rate. When the first circuitry 210 is closer to the set of optimal operational positions 212, the light source can emit a second color (e.g., orange) and/or can flash at a faster rate. When the first circuitry 210 is at the set of optimal operational positions 212, the light source can emit a third color (e.g., green) and/or can be continually on. For another example, the user interface can present other types of visual cues (e.g., a scale comprising a number of bars representing the strength of the coupling or the quality of alignment between the first circuitry 210 and the second circuitry 320). In certain implementations, a plurality of light emitting diodes can be arranged in an array to display arrows indicative of the magnitude and/or direction in which the first circuitry 210 is to be moved towards the set of optimal operational positions 212.

For another example, the user interface can comprise a sound source (e.g., speaker) configured to emit tones and/or to pulse at rates indicative of the magnitude and/or direction. When the first circuitry 210 is farther from the set of optimal operational positions 212, the sound source can emit a first tone (e.g., lower pitch) and/or can pulse at a slow rate. When the first circuitry 210 is closer to the set of optimal operational positions 212, the sound source can emit a second tone (e.g., higher pitch) and/or can pulse at a faster rate. When the first circuitry 210 is at the set of optimal operational positions 212, the sound source can emit a third tone (e.g., highest pitch) and/or can be continually on. In certain implementations in which the apparatus 200 comprises an auditory prosthesis, the user interface can comprise a stimulator unit 120 of the implanted device 300 configured to provide the audio indicia to the recipient.

For another example, the user interface can comprise a vibrator (e.g., haptic motor) configured to emit different vibration waveforms indicative of the magnitude and/or direction. When the first circuitry 210 is farther from the set of optimal operational positions 212, the sound source can emit a first tone (e.g., lower pitch) and/or can pulse at a slow rate. When the first circuitry 210 is farther from the set of optimal operational positions 212, the vibrator can emit a first waveform (e.g., smaller vibration magnitude and/or slower vibration rate). When the first circuitry 210 is closer to the set of optimal operational positions 212, the vibrator can emit a second waveform (e.g., larger vibration magnitude and/or faster vibration rate). When the first circuitry 210 is at the set of optimal operational positions 212, the vibrator can emit a third waveform (e.g., largest vibration magnitude and/or fastest vibration rate).

In certain implementations, the third circuitry 230 is within the component 202 and/or the implanted device 300 and is configured to wirelessly transmit the at least one signal to a communication device separate from the apparatus 200. The communication device can be configured to generate, in response to the at least one signal, audio, visual, or haptic indicia to communicate the magnitude and/or direction to the user (e.g., the example audio, visual, or haptic indicia described above). The communication device can comprise a smartphone, smart tablet, smart watch, or other remote device operated by the user and configured to be in wireless communication with the apparatus 200 (e.g., WiFi; Bluetooth; cellphone connection; telephony; other Internet connection). In certain implementations in which the communication device comprises a viewscreen or other display device, the visual indicia can comprise at least one image (e.g., arrow; scale; diagram; schematic) displayed on the viewscreen. In certain other implementations, at least a portion of the third circuitry 230 is within a device separate from both the component 202 and the implanted device 300 (e.g., smartphone, smart tablet, smart watch, or other remote device operated by the user) and is configured to receive signals indicative of the at least one parameter (e.g., from the component 202 and/or the implanted device 300) and to detect whether the at least one parameter is indicative of the first circuitry 210 being at or displaced from the set of optimal operational positions.

In certain implementations, the third circuitry 230 is configured to compare measured values of the at least one parameter to a set of optimal operational values of the at least one parameter, the set of optimal operational values corresponding to the set of optimal operational positions 212 of the first circuitry 210 relative to the second circuitry 320. For example, one or more initial measured values (e.g., readings) of the at least one parameter can be obtained while the first circuitry 210 is known to be at the set of optimal operational positions 212 (e.g., the inductive communication coils 130, 136 known to be aligned with one another), and the one or more initial measured values can be stored in the storage circuitry as data indicative of the set of optimal operational values. Later, while the component 202 is being positioned over the implanted device 300 by the user, the third circuitry 230 can obtain measured values of the at least one parameter, compare these measured values to the stored data indicative of the set of optimal operational values, and generate guidance information for the user to use to move the component 202 into proper operational positioning relative to the implanted device 300. In certain such implementations, the storage circuitry also contains data (e.g., lookup tables; calculation algorithms) that relate measured values of the at least one parameter to corresponding magnitudes and/or directions for moving the first circuitry 210 towards the set of optimal operational positions 212.

In certain other implementations, the third circuitry 230 is configured to prompt the user (e.g., recipient; practitioner) to move the component 202 among a plurality of positions relative to the implanted device 300 while measuring values of the at least one parameter and to determine the set of optimal operational positions 212 from the measured values. The user can be guided to establish an “ideal” alignment baseline to which subsequent measures of alignment can be compared and corresponding feedback information can be provided to the user.

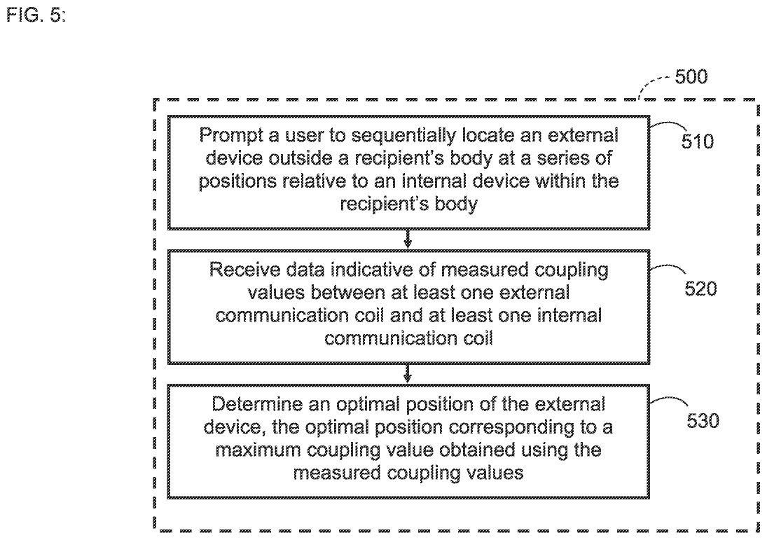

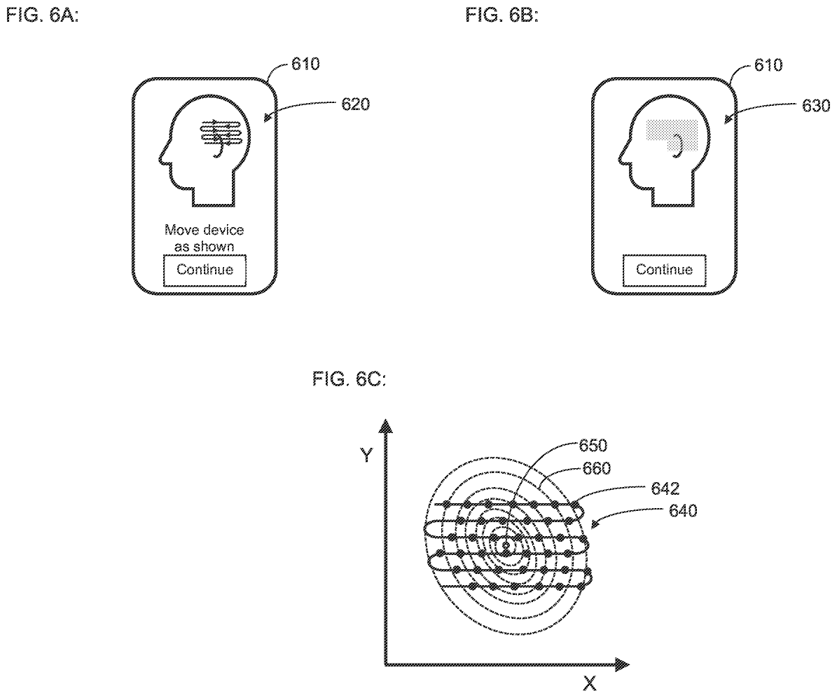

FIG. 5 is a flow diagram of an example method 500 in accordance with certain implementations described herein. While the method 500 is described by referring to some of the structures of the example apparatus 200 of FIGS. 2, 3A-3C, and 4, other apparatus and systems with other configurations of components can also be used to perform the method 500 in accordance with certain implementations described herein. For example, some or each of the operational blocks of the method 500 can be performed using an external device configured to be operationally coupled with an implanted device and/or some or each of the operational blocks of the method 500 can be performed using a separate device in wireless communication with the external device (e.g., device comprising the viewscreen; smartphone; smart tablet; smart watch, or other remote device). FIGS. 6A and 6B schematically illustrate example images shown on a user interface of a communication device visible to the user during the method 500 in accordance with certain implementations described herein. FIG. 6C schematically illustrates a baseline mapping using the data received during the method 500 in accordance with certain implementations described herein. While the method 500 of FIGS. 5 and 6A-6C is described in the context of aligning an external device with a previously-implanted internal device, in certain other implementations, the method 500 can be performed by a practitioner (e.g., surgeon) during a surgical implantation of the internal device whereby the external device is a testing device configured to be inserted into the surgical site to wirelessly coupled to the internal device for testing the performance of the internal device.

In an operational block 510, the method 500 comprises prompting a user to sequentially locate an external device (e.g., component 202) outside a recipient's body at a series of positions relative to an internal device (e.g., implanted device 300) within the recipient's body. The external device (e.g., a sound processor of an auditory prosthesis system) can comprise at least one external communication coil (e.g., first circuitry 210) and the internal device (e.g., implanted stimulation assembly of the auditory prosthesis system) can comprise at least one internal communication coil (e.g., second circuitry 320). For example, the user interface can prompt the user to move the external device around the approximate implant location whilst measuring the at least one parameter indicative of the displacement (e.g., the RF link efficiency), thereby performing a “mapping” of the recipient's body which can be used as a baseline for subsequent positioning of the external device.

As shown in FIG. 6A, a viewscreen 610 can display an image 620 of a head with information (e.g., arrows; text) prompting the user to move the external device to various positions in proximity to the internal device. As shown in FIG. 6B, upon the user moving the external device as prompted, the viewscreen 610 can display an image 630 in real-time with indicia (e.g., shading) indicative of the various positions of the external device so far in the process. As the user continues to move the external device to more positions, the image 630 can be updated, thereby providing the user with feedback while the external device is being moved. The images 620, 630 can comprise a schematic or generic figure of the body portion or an overlay of a camera image/video of the body portion (e.g., augmented reality on a mobile camera).

In an operational block 520, the method 500 further comprises receiving data 640 indicative of measured coupling values between the at least one external communication coil and the at least one internal communication coil. For example, the data 640 can include values indicative of the coupling that have been measured while the at least one external communication coil is located at each of the different positions of the series of positions. For example, the external device can perform link quality measurements while the external device is moved. Examples of values that can be measured include but are not limited to: internal device power and/or voltage values; external device power and/or voltage values; number and/or rate of errors in wireless data transmissions between the external device and the internal device. The data 640 can also include position information indicative of the position of the external device at which the values have been measured (e.g., while the external device performs the link quality measurements). For example, as shown in FIG. 6C, the data 640 can comprise x- and y-coordinates of the external device as the user moves the external device (shown in FIG. 6C as value datapoints 642 located at x- and y-coordinates at which the values are measured). Examples of sensors that can generate the position information include but are not limited to: sensors on or within the external device (e.g., accelerometers; gyroscopes; magnetic field sensors; optical sensors sensitive to visible light and/or non-visible light; acoustic sensors), sensors separate from the external device (e.g., computer vision and/or image recognition sensors; mobile cameras), and combinations thereof.

In an operational block 530, the method 500 further comprises determining at least one optimal position 650 of the external device (e.g., a set of optimal operational positions 212). The at least one optimal position 650 can correspond to a maximum value of the measured values. For example, as shown in FIG. 6C, the external device or other device being used to perform the method 500 can derive, in response to the data 640, a map comprising contour lines 660 (shown as dashed lines in FIG. 6C), each of which traces the x- and y-coordinates of measured values that are substantially equal to one another. Each of the contour lines 660 can be derived (e.g., interpolated) from two or more of the value datapoints 642 of the data 640. In addition, the map can include the at least one optimal position 650 (shown in FIG. 6C as an open circle), which can be derived (e.g., interpolated; obtained; determined) from the various value datapoints 642 and/or the derived contour lines 660. In certain implementations, the data 640, the at least one optimal position 650, and/or the contour lines 660 can be displayed by the user interface (e.g., to indicate to the user where the optimal link quality is located, and therefore where the implant device is to be located).

In certain implementations, the baseline mapping can be performed by a practitioner during an initial fitting procedure of the apparatus 200 to the recipient, while in certain other implementations, the baseline mapping can be performed at various times while the recipient wearing the apparatus 200. In certain such implementations, the method 500 can further comprise triggering the prompting of the operational block 510 (e.g., and of the other operational blocks 520, 530) when the external device is being placed on the recipient's body. For example, the triggering can automatically occur upon the external device being moved from a first position having less than a predetermined threshold coupling value to a second position having greater than the predetermined threshold coupling value (e.g., the external device being place sufficiently close to the implanted device). For another example, the triggering can automatically occur upon a wireless communication link (e.g., Bluetooth connection) being established or reestablished between the external device and the internal device. For still another example, the triggering can automatically occur after a predetermined period of time has elapsed. In other examples, the triggering can occur upon receipt of an input signal from the user, the input signal indicating that the user wishes to refresh the determination of the optimal position (e.g., manually triggered by the recipient). The triggering input signal can come from an input to either the external device or to a user interface of a separate device (e.g., smartphone, smart tablet, smart watch, or other remote device) in wireless operative communication with the external device.

In certain implementations in which the data 640, the at least one optimal position 650, and/or the contour lines 660 are used as a baseline for subsequent positioning of the external device, the user interface can provide real-time (e.g., live) guidance regarding where the external device is to be placed for optimal link quality. In certain implementations, the real-time guidance utilizes information regarding the real-time position of the external device. For example, the position can be monitored in real-time by one or more sensors that include but are not limited to: sensors on or within the external device (e.g., accelerometers; gyroscopes; magnetic field sensors; optical sensors sensitive to visible light and/or non-visible light; acoustic sensors), sensors separate from the external device (e.g., computer vision and/or image recognition sensors; mobile cameras), and combinations thereof.

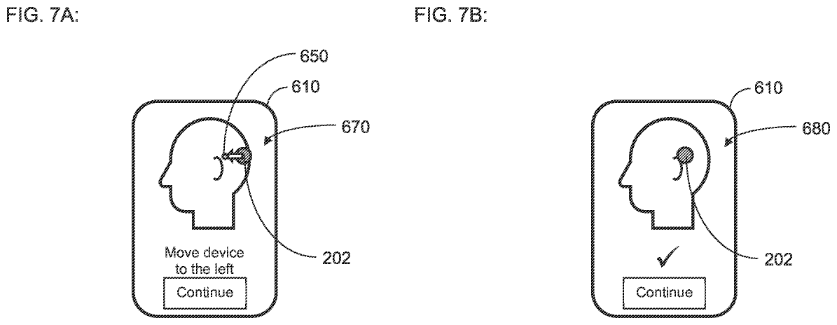

FIGS. 7A and 7B schematically illustrate example images shown on a user interface (e.g., viewscreen 610) of a communication device visible to the user while positioning the external device in accordance with certain implementations described herein. While the external device (e.g., component 202) is displaced from the at least one optimal position 650, as shown in FIG. 7A, the user interface can display an image 670 comprising representations of the recipient's body, the external device, and/or the internal device and indicia (e.g., arrows; text) that are indicative of a magnitude and/or direction for moving the external device (e.g., the component 202 comprising the first circuitry 210) towards the at least one optimal position 650. Upon the external device being located at the at least one optimal position 650, as shown in FIG. 7B, the user interface can display an image 680 comprising representations of the recipient's body, the external device, and/or the internal device and indicia (e.g., text; symbols) that are indicative of successful positioning of the external device to be directly overlying and optimally coupled with the internal device.

In certain implementations, measured data indicative of the alignment during and/or after performing the alignment procedure (e.g., data regarding the recipient's coupling) can be stored (e.g., logged) to inform the recipient's health care provider, compared to such data for other recipients, and used to generate guidance (e.g., recommendations) for improving the recipient's link integrity and efficiency and/or to alter the recipient's care (e.g., by acting upon recommendations for clinical intervention).

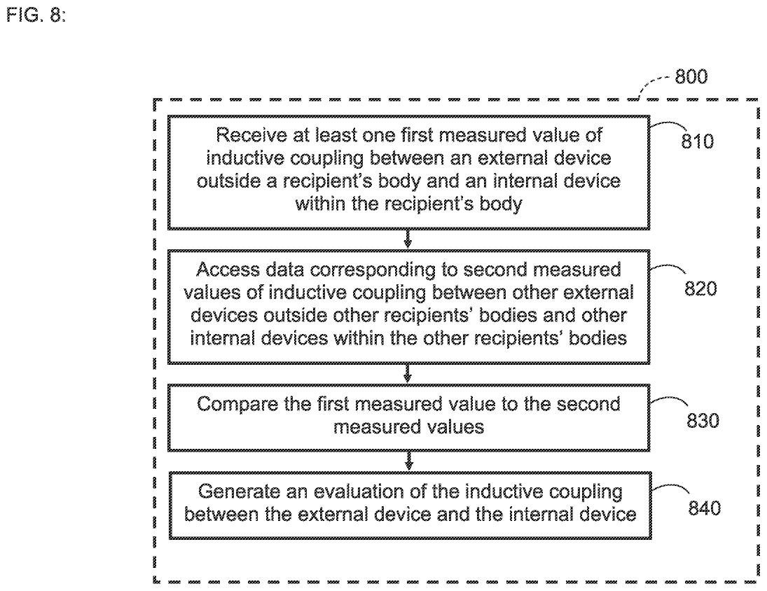

FIG. 8 is a flow diagram of an example method 800 in accordance with certain implementations described herein. While the method 800 is described by referring to some of the structures of the example apparatus 200 of FIGS. 2, 3A-3C, and 4, other apparatus and systems with other configurations of components can also be used to perform the method 800 in accordance with certain implementations described herein. For example, some or each of the operational blocks of the method 800 can be performed using an external device configured to be operationally coupled with an implanted device and/or some or each of the operational blocks of the method 800 can be performed using a separate device in wireless communication with the external device (e.g., device comprising the viewscreen; smartphone; smart tablet; smart watch, or other remote device).

In an operational block 810, the method 800 comprises receiving at least one first measured value of inductive coupling between an external device (e.g., component 202) outside a recipient's body and an internal device (e.g., implanted device 300) within the recipient's body. For example, while the user (e.g., recipient; practitioner) is positioning the component 202 over the implanted device 300, the third circuitry 230 can obtain first measured values of the at least one parameter (e.g., which is indicative of the inductive coupling between the component 202 and the implanted device 300).

In an operational block 820, the method 800 further comprises accessing data corresponding to second measured values of inductive coupling between other external devices outside other recipients' bodies and other internal devices within the other recipients' bodies. For example, such data can be stored at a central database and accessed (e.g., by the external device or by a separate device operated by the user) via communications via the Internet. This data can provide a baseline for optimal positioning derived from the experiences of other recipients having the same or similar characteristics as the recipient (e.g., type of implanted device; skin flap thickness or SFT). In an operational block 830, the method 800 further comprises comparing the first measured value to the second measured values. For example, the second measured values can correspond to the optimal coupling strength of the other recipients and said comparing can use the second measured values as a baseline (e.g., an optimal coupling strength to be achieved by the recipient) for placement of the external device. In an operational block 840, the method 800 further comprises generating, in response to said comparing, an evaluation of the inductive coupling between the external device and the internal device of the recipient. For example, upon the comparison indicating that the inductive coupling between the recipient's external device and internal device is outside an acceptable range as compared to the baseline, the evaluation can comprise guidance information is to be generated, and such guidance information can be provided to the user to facilitate the proper positioning of the external device.

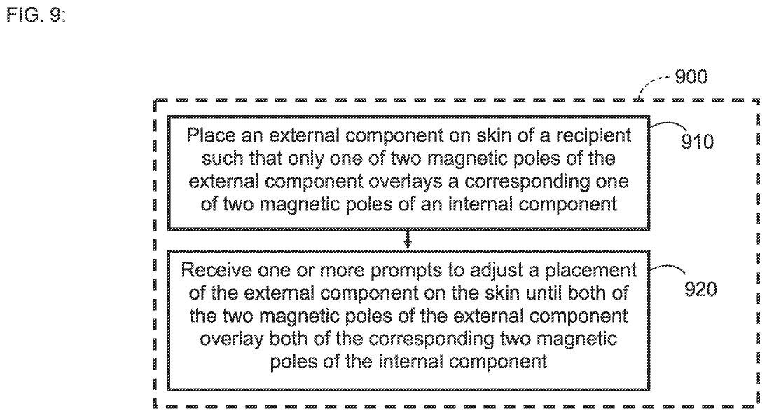

FIG. 9 is a flow diagram of another example method 900 in accordance with certain implementations described herein. While the method 900 is described by referring to some of the structures of the example apparatus 200 of FIGS. 2, 3A-3C, and 4, other apparatus and systems with other configurations of components can also be used to perform the method 900 in accordance with certain implementations described herein. For example, some or each of the operational blocks of the method 900 can be performed using an external device configured to be operationally coupled with an implanted device and/or some or each of the operational blocks of the method 900 can be performed using a separate device in wireless communication with the external device (e.g., device comprising the viewscreen; smartphone; smart tablet; smart watch, or other remote device).

In an operational block 910, the method 900 comprises placing an external component (e.g., component 202) of a medical device on skin 150 of a recipient such that only one of two magnetic poles of the external component overlays a corresponding one of two magnetic poles of an internal component (e.g., implanted device 300) of the medical device implanted in the recipient.

In an operational block 920, the method 900 further comprises receiving one or more prompts originating from the medical device to adjust a placement of the external component on the skin of the recipient until both of the two magnetic poles of the external component overlay both of the corresponding two magnetic poles of the internal component. In certain implementations, the one or more prompts are based on one or more measured values of inductive coupling between the external component and the internal component (e.g., between first circuitry 210 and second circuitry 320).

In certain implementations, having both of the two magnetic poles of the external component overlaying both of the corresponding two magnetic poles of the internal component generates a first retention force configured to retain the external component on the skin of the recipient. In certain implementations, having only one of the two magnetic poles of the external component overlaying the corresponding one of the two magnetic poles of the internal component generates a second retention force less than the first retention force.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having wireless communication between an implanted device and an external device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within +10% of, within +5% of, within +2% of, within +1% of, or within +0.1% of the stated amount. As another example, the terms “generally parallel” and “substantially parallel” refer to a value, amount, or characteristic that departs from exactly parallel by +10 degrees, by +5 degrees, by +2 degrees, by +1 degree, or by +0.1 degree, and the terms “generally perpendicular” and “substantially perpendicular” refer to a value, amount, or characteristic that departs from exactly perpendicular by +10 degrees, by +5 degrees, by +2 degrees, by +1 degree, or by +0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a component configured to be placed over a portion of skin of a recipient, the portion of skin overlaying an implanted device;
first circuitry within the component, the first circuitry configured to wirelessly communicate with second circuitry within the implanted device, the first circuitry having a set of optimal operational positions relative to the second circuitry; and
third circuitry configured to detect at least one parameter indicative of a displacement of the first circuitry from the set of optimal operational positions and to generate at least one signal indicative of a magnitude and/or direction for moving the first circuitry towards the set of optimal operational positions.

2. The apparatus of claim 1, wherein the apparatus further comprises at least one first magnet mounted within the component and configured to generate an attractive magnetic force with at least one second magnet within the implanted device, the attractive magnetic force configured to hold the component on the portion of skin with the first circuitry at the set of optimal operational positions.

3. The apparatus of claim 2, wherein the at least one first magnet comprises a first magnetic moment extending substantially parallel to the portion of skin when the first circuitry is at the set of optimal operational positions, the at least one second magnet comprises a second magnetic moment extending substantially parallel to the portion of skin and configured to rotate within a plane substantially parallel to the portion of skin.

4. The apparatus of claim 2, wherein the at least one second magnet comprises a diametric magnet.

5. The apparatus of claim 2, wherein the at least one parameter comprises a magnetic field strength of the attractive magnetic force.

6. The apparatus of claim 1, wherein the at least one parameter is indicative of efficiency and/or data integrity of communications between the first circuitry and the second circuitry.

7. The apparatus of claim 1, wherein the first circuitry comprises a first inductive communication coil and the second circuitry comprises a second inductive communication coil, the first inductive communication coil configured to be in inductive communication with the second inductive communication coil.

8. The apparatus of claim 7, wherein the at least one parameter comprises a mutual inductance of the first inductive communication coil and the second inductive communication coil.

9. The apparatus of claim 1, wherein the at least one parameter is selected from a group consisting of: a number or rate of communication errors between the first circuitry and the second circuitry; a power consumption rate of communications between the first circuitry and the second circuitry; a voltage or decay rate received by the implanted device.

10. The apparatus of claim 1, the apparatus further comprising a user interface configured to generate audio, visual, or haptic indicia, in response to the at least one signal, to communicate the magnitude and/or direction to a user.

11. The apparatus of claim 1, wherein the third circuitry is within the component and/or the implanted device and is configured to wirelessly transmit the at least one signal to a communication device separate from the apparatus, the communication device configured to generate, in response to the at least one signal, audio, visual, and/or haptic indicia to communicate the magnitude and/or direction to a user.

12. The apparatus of claim 1, wherein the third circuitry is within a device separate from both the component and the implanted device.

13. The apparatus of claim 1, wherein the third circuitry is configured to compare measured values of the at least one parameter to a set of optimal operational values of the at least one parameter, the set of optimal operational values of the at least one parameter corresponding to the set of optimal operational positions of the first circuitry relative to the second circuitry.

14. The apparatus of claim 13, wherein the third circuitry comprises storage circuitry configured to contain data that relate measured values of the at least one parameter to corresponding magnitudes and/or directions for moving the first circuitry towards the set of optimal operational positions.

15. The apparatus of claim 1, wherein the component comprises an external portion of an auditory prosthesis system and the implanted device comprises an internal portion of the auditory prosthesis system.

16. A method comprising:
prompting a user to sequentially locate an external device outside a recipient's body at a series of positions relative to an internal device within the recipient's body, the external device comprising at least one external communication coil and the internal device comprising at least one internal communication coil;
receiving data indicative of measured coupling values between the at least one external communication coil and the at least one internal communication coil, the measured coupling values measured with the at least one external communication coil located at different positions of the series of positions; and
determining an optimal position of the external device, the optimal position corresponding to a maximum coupling value obtained using the measured coupling values.

17. The method of claim 16, further comprising triggering said prompting upon the external device being moved from a first position having less than a predetermined threshold coupling value to a second position having greater than the predetermined threshold coupling value.

18. The method of claim 16, further comprising triggering said prompting upon a wireless communication link being established or reestablished between the external device and the internal device.

19. The method of claim 16, further comprising triggering said prompting upon receipt of an input signal from the user.

20. The method of claim 16, wherein the external device comprises a sound processor of an auditory prosthesis system and the internal device comprises an implanted stimulation assembly of the auditory prosthesis system.

21. A method comprising:
receiving at least one first measured value of inductive coupling between an external device outside a recipient's body and an internal device within the recipient's body;
accessing data corresponding to second measured values of inductive coupling between other external devices outside other recipients' bodies and other internal devices within the other recipients' bodies;
comparing the first measured value to the second measured values; and
generating, in response to said comparing, an evaluation of the inductive coupling between the external device and the internal device.

22.-24. (canceled)

25. A system comprising:
a component having an outer surface portion configured to be placed over a portion of skin of a recipient, the portion of skin overlaying an implanted device;
a first magnet within the component, the first magnet comprising a first substantially flat magnetic surface configured to be substantially parallel to the portion of skin and to have a first north magnetic pole region and a first south magnetic pole region, the implanted device comprising a second magnet having a second substantially flat magnetic surface substantially parallel to the portion of skin and to have a second north magnetic pole region and a second south magnetic pole region; and
circuitry configured to detect a configuration of the first magnet and the second magnet upon the component being placed at least partially over the implanted device and to generate at least one signal indicative of the configuration.

26.-30. (canceled)

31. A method comprising:
placing an external component of a medical device on skin of a recipient such that only one of two magnetic poles of the external component overlays a corresponding one of two magnetic poles of an internal component of the medical device implanted in the recipient; and
receiving one or more prompts originating from the medical device to adjust a placement of the external component on the skin of the recipient until both of the two magnetic poles of the external component overlay both of the corresponding two magnetic poles of the internal component.

32.-34. (canceled)
